(19) United States
(12) Patent Application Publication
(10) Pub. No.: US 2003/0220301 A1
(43) Pub. Date: Nov. 27, 2003

(54) Metformin salts of lipophilic acids

(75) Inventors: Manjari Lal, Bellevue, WA (US); Nagesh Palepu, Mill Creek, WA (US); Dean Kessler, Edmonds, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(73) Assignee: Sonus Pharmaceuticals, Inc.

(21) Appl. No.: 10/369,347

(22) Filed: Feb. 14, 2003

Related U.S. Application Data
(60) Provisional application No. 60/357,196, filed on Feb. 14, 2002.

(51) Int. Cl. A61K 31/665; A61K 31/355; A61K 31/205; C07D 311/74; C07C 279/26

(52) U.S. Cl. 514/101; 514/458; 514/554; 549/410; 562/493; 562/582; 562/590

(57) ABSTRACT

Metformin salts of lipophilic acids, their pharmaceutical formulations, and methods of administrating the metformin salts for the treatment of hyperglycemia.

### CROSS-REFERENCE TO RELATED APPLICATION

The present application claims the benefit of U.S. Provisional Patent Application No. 60/357,196, filed Feb. 14, 2002, which is incorporated herein by reference in its entirety.

### FIELD OF THE INVENTION

The present invention relates to metformin salts of lipophilic acids, formulations including metformin salts of lipophilic acids, and methods for administering metformin salts of lipophilic acids.

### BACKGROUND OF THE INVENTION

Metformin is a biguanide, anti-hyperglycemic agent currently marketed in the United States in the form of its hydrochloride salt (GLUCOPHAGE, Bristol-Myers Squibb Company). The oral medication is designed to help control elevated blood sugar levels in NIDDM (non-insulin-dependent diabetes mellitus) or Type II diabetes. Current metformin therapy has proven less than optimal as it is associated with a high incidence of gastrointestinal side effects. Further, the drug is commonly administered at high doses (as oral tablets) 2 or 3 times per day to achieve effective glucose-lowering treatment. Anonymous, “Glucophage Prescription Information,” Bristol-Myers Squibb Company, Princeton, N.J., 1999.

Metformin hydrochloride is highly water soluble with a pKa=12.4. The drug's absorption pattern is affected by ionized metformin's tendency to adsorb to the negatively charged intestinal epithelium. Swift renal elimination and without significant metabolism is caused by the high polarity of the drug. D. Stepensky, et al., “Preclinical Evaluation of Pharmacokinetic-Pharmacodynamic Rationale for Oral CR Metformin Formulation,” J. Cont. Release 71:107-115, 2001. Studies have indicated that metformin has poor colonic absorption in healthy human subjects. N. Vidon, et al., “Metformin in the digestive tract,” Diabetes Res. Clin. Pract. 4:223-229, 1988; P. H. Marathe, et al., “Effect of Altered Gastric Emptying and Gastrointestinal Motility on Bioavailability of Metformin,” AAPS Annual Meeting, New Orleans, La., 1999.

Metformin hydrochloride is not readily absorbed throughout the entirety of the gastrointestinal tract due, at least in part, to its extremely high water solubility and absorbs only in the duodenal region of the small intestine. One way of improving the bioavailability of metformin is by retaining the drug in the stomach for a longer time and releasing the drug slowly from the tablet matrix retained in the stomach. This type of dosage form is to referred as a gastro-retentive tablet. Disadvantages of this dosage form include (1) highly variable absorption; (2) residential time in the stomach is high; and (3) the limited area for absorption necessitates multiple dosing per day.

Metformin absorption is saturable and incomplete. At the usual metformin doses and dosing schedules, steady-state plasma concentrations are reached within 24 to 48 hours and are generally less than 1 ug/mL. In controlled clinical trials, maximum metformin plasma levels (Cₘₐₓ) did not exceed 4 ug/mL, even at maximum doses.

The present invention seeks to overcome these disadvantages by providing a formulation of metformin hydrochloride in a controlled release system wherein the drug may be administered in lower doses.

### SUMMARY OF THE INVENTION

In one aspect, the present invention provides metformin salts of lipophilic acids. The metformin salts of the invention are highly lipophilic and exhibit enhanced absorption of metformin to provide for improved uptake of the drug throughout the entire GI tract and enable sustained control of blood glucose levels. The metformin salts of the invention have anti-hyperglycemic activity and can be used as anti-hyperglycemic agents. The metformin salts of the invention include positively charged metformin and a suitable negatively charged lipophilic acid. Suitable lipophilic acids include tocopherol acid derivatives and fatty acids.

In other aspects, the invention provides pharmaceutical formulations of the metformin lipophilic acid salts. In one embodiment, the formulation is a biocompatible gel for controlled release of metformin.

In another aspect of the invention, methods for making the metformin salts and their formulations are provided.

In a further aspect, the invention provides a method for treating hyperglycemia through the administration of the metformin salt formulations.

### BRIEF DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a table summarizing blood glucose levels after metformin salt dose (mg/dL) as a function of time for metformin hydrochloride and representative metformin salts of the invention;

FIG. 2 is a table summarizing percent change in blood glucose levels after metformin salt dose (mg/dL) as a function of time for metformin hydrochloride and representative metformin salts of the invention; and

FIG. 3 is a graph illustrating the percent change in blood glucose levels after metformin salt dose (mg/dL) as a function of time for metformin hydrochloride and representative metformin salts of the invention.

FIG. 4 is a table summarizing the pharmacokinetic parameters of metformin following metformin salt dose (mg/kg) administration via an oral, intraduodenal, or colonic route for metformin hydrochloride and representative metformin salts of the invention.

FIG. 5 is a graph illustrating the change in blood levels of metformin (μg/mL) after oral administration of metformin salt dose (mg/kg) as a function of time for metformin hydrochloride and representative salts of the invention.

FIG. 6 is a graph illustrating the change in blood levels of metformin (μg/mL) after intraduodenal administration of metformin salt dose (mg/kg) as a function of time for metformin hydrochloride and representative metformin salts of the invention.

FIG. 7 is a graph illustrating the change in blood levels of metformin (μg/mL) after colonic administration of metformin salt dose (mg/kg) as a function of time for metformin hydrochloride and representative metformin salts of the invention.

### DETAILED DESCRIPTION OF THE PREFERRED EMBODIMENT

The following definitions are provided for the purpose of clarifying the terms as they are used in the specification and claims to describe the invention.

The term “metformin” refers to a biguanide oral anti-hyperglycemic agent that is commercially available from Bristol-Myers Squibb Company in the form of its hydrochloride salt GLUCOPHAGE. Metformin hydrochloride (N,N-dimethylimidodicarbonimidic diamide hydrochloride) has a molecular formula of C₄H₁₁N₅ .HCl and a molecular weight of 165.63. Metformin hydrochloride is a cohesive white powder that is highly soluble in water (>300 mg/ml at ambient temperature), has a hygroscopicity measured at 95% relative humidity (25° C.) of greater than 20% moisture uptake at 6 hours, and a high compaction susceptibility.

The term “lipophilic” refers to compounds that have greater solubility in oil than in aqueous medium, and the term “lipopophilic acids” includes tocopherol acid derivatives and fatty acids.

The term “tocopherol” refers to tocopherol compounds including α-tocopherol, β-tocopherol, γ-tocopherol, δ-tocopherol, α-tocotrienol, β-tocotrienol, γ-tocotrienol, and δ-tocotrienol.

The term “pharmaceutically-acceptable carrier” as used herein means one or more compatible solid or liquid fillers, dilutants, or encapsulating substances that are suitable for administration to a human or other animal.

The term “carrier” refers to an organic or inorganic ingredient, natural or synthetic, with which the metformin lipophilic acid salt is combined to facilitate the application.

The term “parenteral” refers to subcutaneous, intravenous, intramuscular, or infusion delivery.

The term “biocompatible” refers to a substance that has no medically unacceptable toxic or injurious effects on biological function.

The term “therapeutically effective amount” refers to an optimized amount of metformin lipophilic salt such that the desired antihyperglycemic activity is provided without significant side-effects. The precise dosage level should be determined by the attending physician or other health care provider and will depend upon well-known factors, including route of administration, and the age, body weight, sex and general health of the individual; and the use (or not) of concomitant therapies. Of course, the skilled person will realize that divided and partial doses are also within the scope of the invention.

The term “hyperglycemia” refers to an elevation of glucose levels in the blood beyond the normal range.

The term “antihyperglycemic activity” refers to a determination that fasting plasma glucose and glycoslyated hemoglobin levels are decreased from the pre-treatment hyperglycemic state to normal or near normal levels.

The term “Cₘₐₓ” refers to the peak or maximum concentration of metformin in a defined body compartment (e.g., blood, plasma or serum).

The term “area-under-the-curve” or “AUC” refers to the integral of metformin concentration in the blood over time, from zero to infinity or any interim time point.

In one aspect, the present invention provides metformin salts of lipophilic acids. The metformin salts of the invention have anti-hyperglycemic activity and can be used as anti-hyperglycemic agents. The metformin salts of the invention are highly lipophilic and provide for enhanced absorption of metformin. The metformin lipophilic acid salts of the invention include positively charged metformin and a suitable negatively charged lipophilic acid. Generally, positively charged metformin is protonated metformin. Suitable lipophilic acids include tocopherol acid derivatives and fatty acids.

In some embodiments, the invention provides metformin salts of tocopherol acid derivatives. Representative tocopherol acid derivatives useful in the practice of this invention include tocopherol carboxylates and tocopherol phosphates. Examples of tocopherol carboxylates include acid esters of tocopherol and polybasic acids (e.g., succinic acid, citraconic acid, methylcitraconic acid, itaconic acid, maleic acid, glutaric acid, glutaconic acid, and phthalic acids). Examples of tocopherol acid esters include tocopherol acid succinate, tocopherol acid citraconate, tocopherol acid methylcitraconate, tocopherol acid itaconate, tocopherol acid maleate, tocopherol acid glutarate, tocopherol acid glutaconate, and tocopherol acid phthalate, among others.

In one embodiment, the lipophilic acid is tocopherol succinate. In another embodiment, the lipophilic acid is tocopherol phosphate.

In other embodiments, fatty acids are suitable lipophilic acids of the present invention. Useful fatty acids in the practice of the invention include naturally occurring, non-naturally occurring, branched or unbranched fatty acids having from about 8 carbon atoms to about 20 carbon atoms. Representative examples of common unbranched naturally occurring fatty acids include C12:0 (lauric acid), C14:0 (myristic acid), C16:0 (palmitic acid), C16:1 (palmitoleic acid), C16:2, C18:0 (stearic acid), C18:1 (oleic acid), C18:1-7 (vaccenic), C18:2-6 (linoleic acid), C18:3-3 (α-linolenic acid), C18:3-5 (eleostearic), C18:3-6 (β-linolenic acid), C18:4-3, C20:1 (gondoic acid), C20:2-6, C20:3-6 (dihomo-y-linolenic acid), C20:4-3, C20:4-6 (arachidonic acid), and C20:5-3 (eicosapentaenoic acid)

In one embodiment, the lipophilic acid is oleic acid. In another embodiment, the lipophilic acid is stearic acid. In another embodiment, the lipophilic acid is lipoic acid (i.e., 6,8-dithiooctanoic acid or thioctic acid).

In another aspect, the present invention provides pharmaceutical formulations. The formulations include one or more metformin lipophilic acid salts in combination with a pharmaceutically-acceptable carrier. The components of the pharmaceutical formulation are capable of being commingled with the salts of the present invention, and with each other, in a manner such that there is no interaction that would substantially impair the desired pharmaceutical efficacy.

Representative examples of pharmaceutically acceptable carriers of the invention include carriers that include salts, buffering agents, preservatives, compatible carriers, solvents, and optionally other therapeutic ingredients.

In addition to the metformin lipophilic acid salt, the pharmaceutical formulation can include a variety of excipients including sterile water, normal saline, D5W, Ringer's solution, or other equivalent infusion liquids. The formulations can also be appropriately modified according to specific treatment schemes adopted by clinicians.

The metformin salts of the invention are potent anti-hyperglycemic agents having substantially equivalent activity compared to metformin hydrochloride as described in Example 5. The metformin salts have the advantage of increased lipophilicity, which improves the absorption of metformin throughout the gastrointestinal tract as described in Example 8.

The metformin salts can be administered in any medically suitable manner as pharmaceutical formulations to various mammalian species, such as dogs, cats, and humans in need of such treatment. Examples of routes of administration include oral, parentaral, intravenous, rectal, intraduodenal, or as a bolus injection. The formulation may vary according to the intended route of administration and may take the form of capsules, liposomes, time delayed coatings, pills, or may be formulated as gels for controlled release.

The method of administration can be as for metformin hydrochloride. The metformin salts of the invention can be administered systemically.

In one embodiment, the formulation is administered orally. Formulations for oral administration can include deionized water, phosphate buffered saline, lyophylized powder in the form of tablets and capsules, and may further include various fillers, binders, and the like.

In another embodiment, the formulation is administered parenterally. In one embodiment, the formulation is administered intravenously. Formulations for injection may include physiologically-acceptable media, such as water, saline, phosphate buffered saline (PBS), aqueous ethanol, aqueous polyethylene glycols, or the like.

The present invention provides gel formulations of metformin salts that exhibit improved absorption of metformin throughout the gastrointestinal tract of an animal or human. The gels of the invention provide a method of controlled release of metformin. The gels may be administered in any medically suitable manner including intraduodenal, colonic, and oral administration. The gels are biocompatible and are not significantly toxic in warm-blooded animals such as humans. The gels of the invention may be lyophilized, stored in a powder form, and subsequently rehydrated into a gel state.

In one embodiment, the gel is a tocopherol phosphate gel. The preparation of a representative tocopherol phosphate gel is described in Example 6. The metformin lipophilic acid salts of the invention may be formulated into a biocompatible gel. In one embodiment, the lipophilic acid is tocopherol phosphoric acid (i.e., tocopherol phosphate). In another embodiment, the lipophilic acid is tocopherol nicotinic acid. Other embodiments can include lipoic acid as well as the fatty acids listed herein. The metformin tocopherol phosphate gel can be lyophilized to form a solid which, when dispersed into an aqueous tocopherol phosphate solution, forms a biocompatible gel. The preparation of two representative metformin/tocopherol phosphate gels is described in Example 7. Example 7 describes the preparation of a metformin hydrochloride/tocopherol phosphate gel and the preparation of metformin tocopherol phosphate/tocopherol phosphate gel. As noted above, tocopherol phosphate gels can include other metformin salts of lipophilic acids.

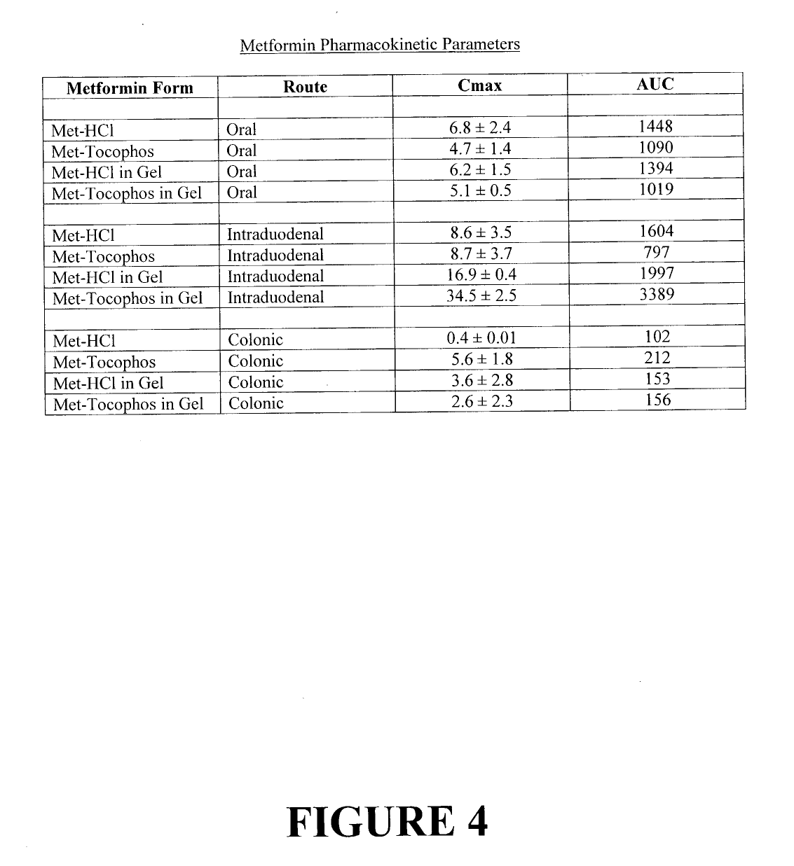

The improved absorption of metformin after administration of the biocompatible gels of the invention including metformin salts of lipophilic acids is described in Example 8. In some embodiments, the metformin salt formulation provides a peak blood concentration (Cₘₐₓ) at least about 4-fold higher than the peak blood concentration of metformin after administration of metformin hydrochloride as shown in FIG. 4 (compare, for example, intraduodenal administration of Met-HCl and Met-Tocophos in gel).

In another aspect of the invention, methods for making the metformin salts of the invention are provided. The preparation of representative metformin salts of the invention including metformin α-tocopherol phosphate, metformin α-tocopherol succinate, and metformin lipoate are described in Examples 1-4. In the methods, metformin hydrochloride is treated with a lipophilic acid in aqueous base to provide the metformin lipophilic acid salt. The product salt can be recovered from the reaction mixture by filtration and dried to provide a free flowing solid.

In a further aspect, the invention provides a method of producing a gel that includes metformin salts (i.e., a metformin gel). In one embodiment, the metformin gel is prepared from an aqueous solution of tocopherol phosphate by the addition of sodium chloride, followed by the addition of metformin hydrochloride. In another embodiment, the gelation is achieved using basic amino acids such as, for example, glycine or arginine. The gelation is dependent on temperature and pH. Gelation was observed in a pH range between 8 and 9. The solution remains a liquid at room temperature and gelled at approximately 37° C. Representative examples of the methods of preparing the metformin gel formulations are described in Example 6 and Example 7.

In another aspect, the invention provides a method for treating hyperglycemia in a warm-blooded animal by administering a metformin salt of the invention. The method can be used to treat hyperglycemia including Type II diabetes (NIDDM) and/or Type I diabetes (IDDM). The method includes providing a pharmaceutical formulation including metformin salt of the invention and a pharmaceutically-acceptable carrier, and administering the pharmaceutical formulation in a therapeutically effective amount to a warm-blooded animal in need thereof.

The dose administered is generally adjusted according to the age, weight, and condition of the patient, taking into account as the route of administration, dosage form and regimen, and the desired result. In general, the dosage forms of the metformin salts of the invention may be administered in amounts as described for metformin hydrochloride (Bristol-Myers Squibb Company's GLUCOPHAGE) as set out in the Physician's Desk Reference. For example, oral dosage of metformin hydrochloride is individualized on the basis of effectiveness and tolerance, while not exceeding the maximum daily recommended dose of 2550 mg in adults and 2000 mg in pediatric patients (Bristol-Myers Prescription information, www.bms.com/medicines/data/). Metformin hydrochloride is typically administered in divided doses with meals, and is generally started at a low dose, usually no lower than 850 mg/day, with gradual escalation to permit identification of the minimum therapeutically effective amount required for adequate anti-hyperglycemic activity.

In another embodiment, the method for treating hyperglycemia of the invention may include administering a metformin salt of the invention in combination with one or more additional therapeutic agents used for the treatment of hyperglycemia. Examples of such therapeutic agents include oral medications such as sulfonylureas, meglitinide, alpha glucosidease inhibitors, and thiazolidinediones.

In summary, the invention provides metformin salts having substantially equivalent anti-hyperglycemic activity compared to metformin hydrochloride and having improved effects on blood glucose levels. Other advantages of the metformin salts of the invention relate to handling properties, including lower hygroscopicity and better flow properties compared to metformin hydrochloride salt. Furthermore, the metformin salts of the invention are significantly less soluble in water than metformin hydrochloride and thus provide the opportunity for formulating metformin in controlled release systems that require less polymer excipients to achieve a desired metformin release rate. By virtue of their lipophilic component, the metformin salts of the invention will have improved gastrointestinal absorption characteristics compared to metformin hydrochloride.

The following examples are provided for the purpose of illustrating, not limiting, the invention.

### EXAMPLES

### Example 1

### Preparation of Metformin Tocopherol Succinate (1:1)

In this example, the preparation of a representative metformin salt of the invention, metformin tocopherol succinate (1:1), is described.

4.0 g (2.41×10⁻² mole) of metformin hydrochloride was dissolved in 20 ml of H₂O. The pH was adjusted to 13.05 with 50% NaOH. With stirring, a solution of 6.2 g (1.20×10⁻² mole) of vitamin E succinic acid (VESA) in 15 ml acetone was added dropwise to the metformin solution while heating at 70° C. Precipitation occurred immediately. After stirring for 10 minutes, the solution cleared, resulting in a yellow, clear, single-phase system. The solution clouded upon cooling. After stirring for 15 hours, the precipitated product was collected by filtration and washed with acetone to yield the product as a yellow-white solid.

### Example 2

### Preparation of Metformin Tocopherol Succinate (1:2)

In this example, the preparation of a representative metformin salt of the invention, metformin tocopherol succinate (1:2), is described.

0.5 g (3.01×10⁻³ mole) of metformin hydrochloride was dissolved in 5 ml of H₂O. The pH was adjusted to 13.05 with 50% NaOH. With stirring, a solution of 3.1 g (6.0×10⁻³ mole) vitamin E succinic acid (VESA) in 15 ml acetone was added dropwise to the metformin solution while heating at 60° C. The solution clouded upon cooling and the solvent removed under reduced pressure to provide the product as a wax.

### Example 3

### Preparation of Metformin Tocopherol Phosphate (1:1)

In this example, the preparation of a representative metformin salt of the invention, metformin tocopherol phosphate, is described.

1.0 g (6.04×10⁻³ moles) of metformin hydrochloride was dissolved in 10 ml H2O (Solution 1). A second solution of 1.67 g (3.01×10⁻³ mole) of tocopherol phosphate (disodium salt) in 30 ml H₂O was prepared from tocopherol phosphate and water by sonication for 90 minutes at 50° C. (or optionally stirred with heat for approximately 4 hours). Solution 2 provided in a clear solution having a pH of 10.6, which was adjusted to 12.9 with 10% NaOH. Solution 2 was then added dropwise to Solution 1 while heating at 60° C. The resulting solution (Solution 3) was stirred for 4-5 hours, removed from heat, and stirred overnight. The solvent was removed by rotary evaporation to yield a yellow, viscous oil. The product that was dried in a vacuum oven overnight to provide a dry powder.

### Example 4

### Preparation of Metformin Lipoate

In this example, the preparation of a representative metformin salt of the invention, metformin lipoate, is described.

To a stirred solution of metformin hydrochloride in water at pH 9.5 was added a solution of thioctic acid (α-lipoic acid) in acetone. The solution clouded upon cooling and removal of solvent gave a yellow-white powder.

### Example 5

### In Vivo Experiments to Evaluate Efficacy of Metformin Tocopherol Salts

In this example, the efficacy of representative metformin tocopherol acid salts for affecting blood glucose levels in vivo is described. The metformin salts were metformin tocopherol succinate; metformin tocopherol phosphate (1:1); metformin tocopherol phosphate (1:2); metformin oleate; and metformin hydrochloride in water except metformin tocopherol succinate, which was dissolved in dimethylsulfoxide.

A study was conducted to compare the glucose lowering effect of the above-noted four representative metformin salts in a rat model of non-insulin-dependent diabetes mellitus (NIDDM). NIDDM was chemically induced in five Sprague-Dawley rats (250-300 grams) by injecting streptozotocin (50 mg/kg) intraperitoneally. Streptozotocin solution was prepared in 0.01 M sodium citrate to a final concentration of 25 mg/ml.

Approximately five days after streptozotocin administration, the venous blood glucose level was assayed with a SURESTEP blood glucose monitor (Lifescan) in unfasted rats. Only animals with blood glucose above 300 mg/dl (unfasted) were used for the experiments.

The following compounds were administered to the rats by oral gavage at a constant dose volume of 9 mL/kg.

| | Metformin | | | |
|---|---|---|---|---|
| | Con- | | Dosage | |
| Compound | centration | | mg/kg | |
| 0.9% Sodium chloride | 0 | mg/mL | 0 | mg/kg |
| Metformin hydrochloride | 13 | mg/mL | 117 | mg/kg |
| Metformin tocopherol | 5 | mg/mL | 45 | mg/kg |
| phosphate (1:2) | | | | |
| Metformin oleate | 26 | mg/ml | 234 | mg/kg |
| Metformin tocopherol | 17 | mg/ml | 153 | mg/kg |
| succinate | | | | |

A venous blood sample was obtained from the tail vein for determination of glucose concentration at the following time points: 0 (pre-treatment), 0.5, 1, 2, 3, 4, 5, 6, 7, 8, 9 and 24 hours post treatment.

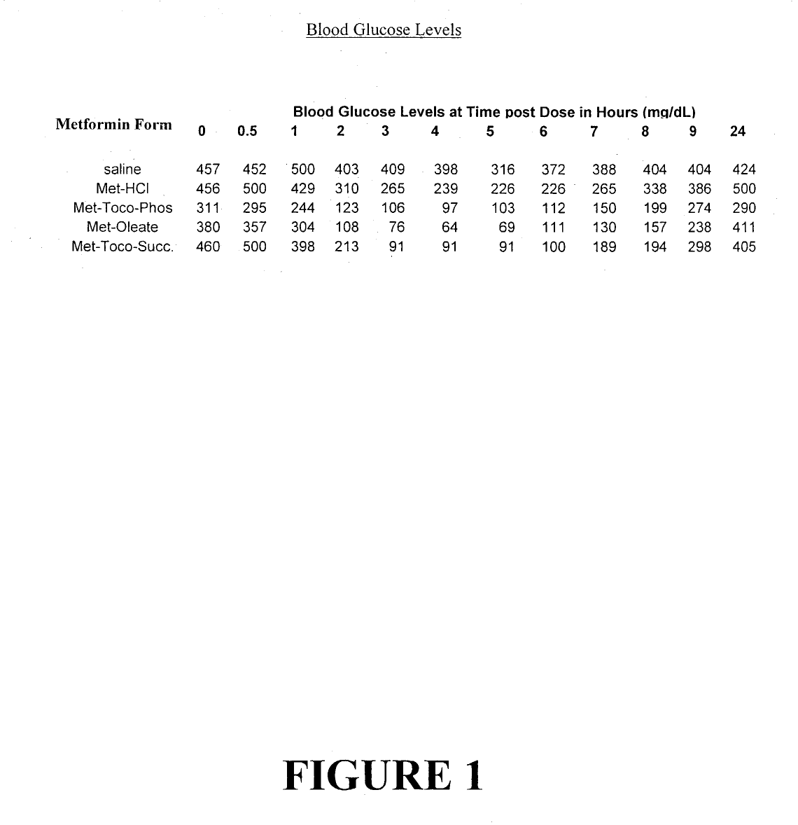

Blood glucose concentration at each time point for each animal is tabulated in FIG. 1. The percent change from baseline was determined for each time point by:

percent change=[blood glucose at time t/blood glucose at time 0]×100%.

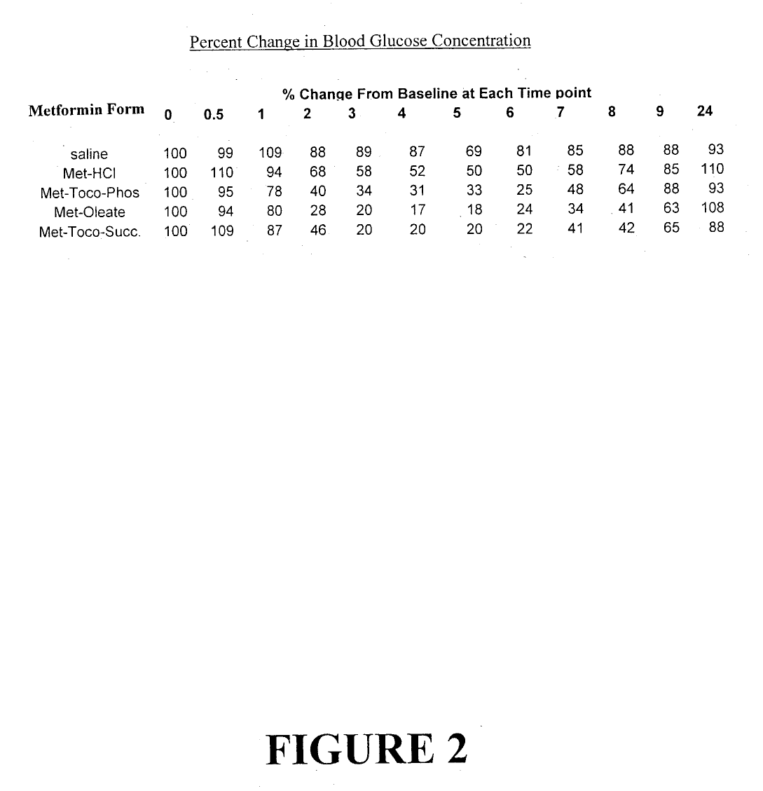

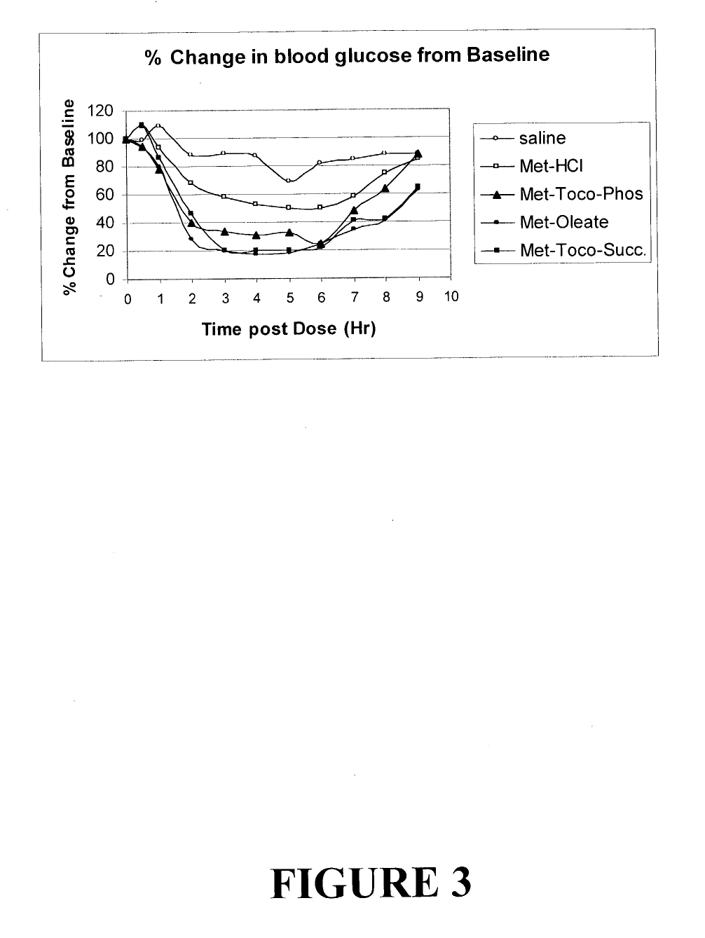

The percent change in glucose concentration is tabulated in FIG. 2 and graphically depicted in FIG. 3.

In the figures, “Met-HCl” and “Metformin HCl” refer to metformin hydrochloride; “Met-Toco-Phos”, “Met-Tocophos”, and “Metformin-Tocophosphate” refer to metformin tocopherol phosphate; “Met-Toco-Succ” refers to metformin tocopherol succinate; and “Met-Oleate” refers to metformin oleate.

The results demonstrate that the metformin salts of the invention were as efficacious as metformin hydrochloride in controlling blood glucose levels in vivo. The metformin salts of the invention have increased lipophilicity, which prolongs the intestinal uptake of metformin thereby providing improved absorption throughout the gastrointestinal tract and provides for improved control of blood glucose levels.

### Example 6

### The Preparation of a Representative Tocopherol Phosphate Gel

In this example, the preparation of a representative tocopherol phosphate gel of the invention is described.

A 3% tocopherol phosphate aqueous solution was prepared by dissolving tocopherol phosphate in water. The resulting solution was clear with a pH of about 11.0. The pH was reduced to pH 8.8 with 1N HCl. With stirring, NaCl (solid) was added to yield a 0.9% NaCl solution which was cloudy without settlement at room temperature (25° C.). The solution was then warmed to 37° C. which provided in a clear solution that was transformed into a transparent gel. The gel remained transparent while the temperature was maintained at 37° C., and returned to a cloudy liquid state at 25° C.

Gelation was also observed when basic amino acids, such as glycine or arginine, were added to the above solution after NaCl addition.

### Example 7

### The Preparation of Representative Metformin Tocopherol Phosphate Gels

In this example, the preparation of two representative metformin tocopherol phosphate gels of the invention are described.

Preparation of metformin hydrochloride/tocopherol phosphate gel. With stirring, a solution of 30 mg/ml metformin hydrochloride in water was added to the aqueous tocopherol phosphate solution (prepared as described in Example 6) at 25° C. Upon addition of metformin hydrochloride, the entire solution formed a transparent gel at 25° C., and the gel remained stable at 37° C.

Preparation of metformin tocopherol phosphate/tocopherol phosphate gel. To the tocopherol phosphate solution prepared as described in Example 6, metformin tocopherol phosphate (solid), prepared as described in Example 3, was added with 15-20 minutes of sonication to disperse the solids. The addition of the metformin tocopherol phosphate transformed the entire solution into a transparent gel at 25° C., and the gel remained stable at 37° C.

Lyophilized tocopherol phosphate gel containing metformin tocopherol phosphate ion pair. Tocopherol phosphate gel (10 g) containing metformin tocopherol phosphate ion pair, prepared as described above, was lyophilized to yield a dry powder. This dry powder reconstituted into a gel upon addition of water, indicating that the tocopherol phosphate gel can be stored as dry powder at room temperature.

### Example 8

### Pharmacokinetic Parameters of Representative Metformin Lipophilic Acid Salts

In this example, the pharmacokinetic parameters of representative metformin lipophilic acid salt formulations is described using oral, intraduodenal, and colonic routes of administration.

A study was conducted to compare the blood levels of metformin over a 24 hour period in a rat model after metformin salt dose (mg/kg) administered in an oral, intraduodenal, or colonic route for metformin hydrochloride and representative metformin salt formulations of the invention. The results for representative metformin lipophilic acid salt formulations of the invention, metformin tocopherol phosphate and metformin tocopherol phosphate/tocopherol phosphate gel, were compared to metformin hydrochloride and metformin hydrochloride/tocopherol phosphate gel.

Metformin hydrochloride and metformin tocopherol phosphate were administered as aqueous formulations.

The number of animals tested for each route of administration is shown in the following table:

| | Number of Animals | | |
|---|---|---|---|
| Formulation | Oral | Intraduodenal | Colonic |
| Metformin hydrochloride | 4 | 2 | 3 |
| Metformin tocopherol phosphate | 4 | 2 | 3 |
| Metformin hydrochloride gel | 4 | 2 | 2 |
| Metformin tocopherol phosphate gel | 3 | 3 | 3 |

All animals were administered a dosage of 100 mg metformin/kg bodyweight. Blood samples were obtained at the following time points: 5, 15, 30, 60, 120, 180, 240, 300 and 360 minutes post administration.

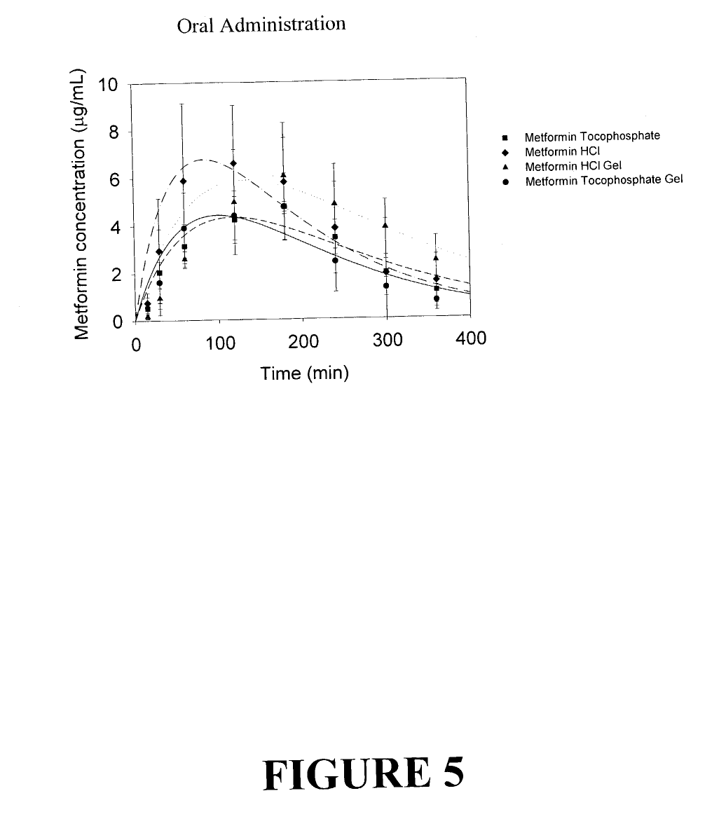

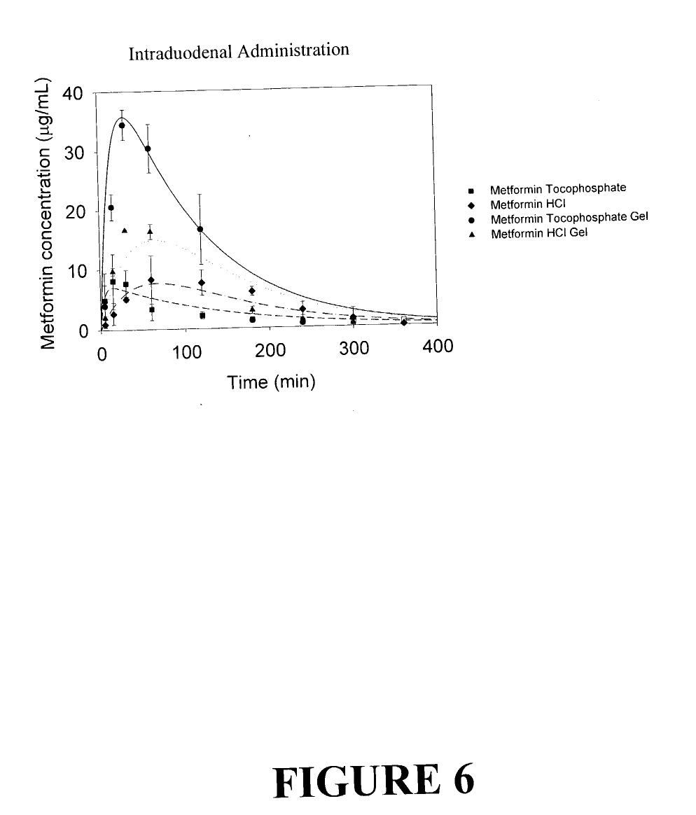

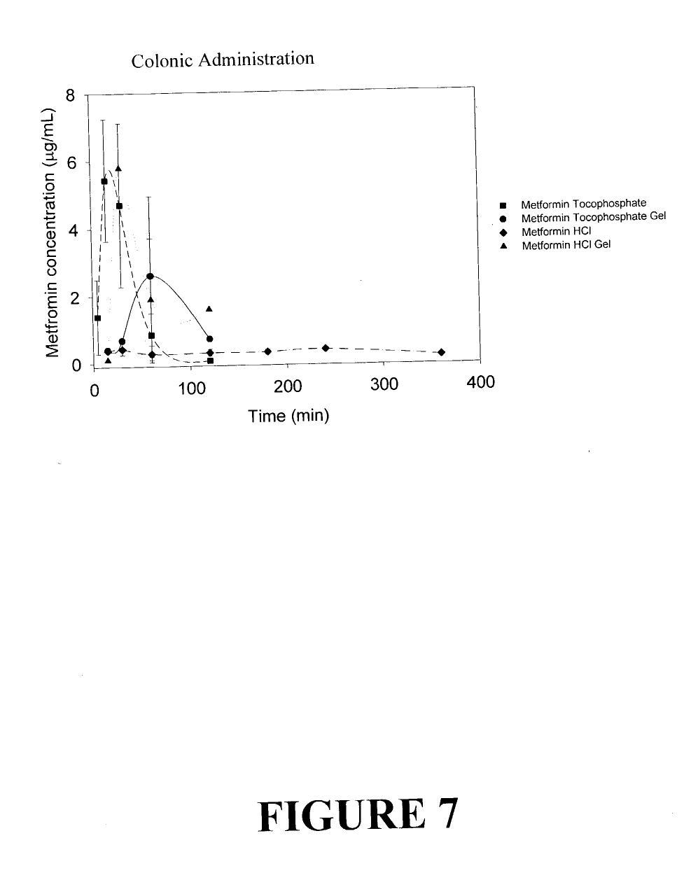

The blood samples were analyzed for metformin concentration by high performance liquid chromatography (HPLC). The results are shown in FIGS. 4-7. FIG. 4 is a table summarizing the pharmacokinetic parameters of metformin following metformin salt dose, expressed as a maximum concentration (Cₘₐₓ) of metformin, and area-under-the concentration-time-curve (AUC). FIGS. 5-7 graphically depict the change in blood metformin levels (μg/mL) after administration of metformin salt dose as a function of time after oral (FIG. 5), intraduodenal (FIG. 6), and colonic (FIG. 7) administration.

The highest level of metformin adsorption was achieved with the metformin tocopherol phosphate gel administered intraduodenally, which resulted in about a 4-fold increase in Cₘₐₓ as compared to metformin hydrochloride as shown in FIG. 4 and FIG. 6. Similarly, the metformin lipophilic salts of the invention were absorbed more readily than metformin hydrochloride after colonic administration, as shown in FIG. 4 and graphically depicted in FIG. 7. The metformin salts were absorbed equally as well as metformin hydrochloride after oral administration, as shown in FIG. 4 and graphically depicted in FIG. 5.

Metformin hydrochloride is not readily absorbed throughout the entirety of the gastrointestinal tract due, at least in part, to its extremely high water solubility. The metformin salts of the invention have increased lipophilicity, which prolongs intestinal uptake. The gel formulations of the invention provide for increased absorption of metformin in the gastrointestinal tract.

### Example 9

### Assessment of Colonic Absorption in Rats (In Situ)

In this example, colonic absorption in rats (in situ) of a representative metformin salt of the invention is compared to metformin hydrochloride.

Because metformin hydrochloride is poorly absorbed in the colonic region of the intestinal tract, it is appropriate to test the hypothesis that a more lipophilic formulation of metformin would be better absorbed in this region. The colon of anesthetized rats (n=2/compound) was cannulated at both ends and purged of all endogenous material. Diluted drug formulations were instilled and samples obtained every 5 minutes for 30 minutes and analyzed for metformin concentration to determine the absorption rate constant (kₐ).

The concentration of metformin in the colon over time was determined for metformin hydrochloride and metformin tocopherol phosphate (1:2). The absorption rate constant was determined to be 0.048 h⁻¹ and 0.054 h⁻¹ for absorption of metaformin hydrochoride. The absorption rate constant was determined to be 1.28 h⁻¹ and 1.15 h⁻¹ for absorption of metaformin tocopherol phosphate (1:2). These rate constants show that metformin tocopherol phosphate (1:2) is absorbed much faster and more extensively than metformin hydrochloride in the colonic region.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A metformin lipophilic acid salt, comprising metformin and a lipophilic acid.

2. The metformin lipophilic acid salt of claim 1, wherein the lipophilic acid is at least one of a tocopherol acid derivative or a fatty acid.

3. The metformin lipophilic acid salt of claim 2, wherein the lipophilic acid is a tocopherol acid derivative.

4. The metformin lipophilic acid salt of claim 3, wherein the tocopherol acid derivative is at least one of a tocopherol carboxylate or a tocopherol phosphate.

5. The metformin lipophilic acid salt of claim 4, wherein the tocopherol carboxylate is at least one of a tocopherol acid ester of a polybasic acid.

6. The metformin lipophilic acid salt of claim 5, wherein the tocopherol acid ester is at least one of tocopherol acid succinate, tocopherol acid citraconate, tocopherol acid methylcitraconate, tocopherol acid itaconate, tocopherol acid maleate, tocopherol acid glutarate, tocopherol acid glutaconate, or tocopherol acid phthalate.

7. The metformin lipophilic acid salt of claim 4, wherein the polybasic acid is at least one of succinic acid, citraconic acid, methylcitraconic acid, itaconic acid, maleic acid, glutaric acid, glutaconic acid, or phthalic acid.

8. The metformin lipophilic acid salt of claim 1, wherein the lipophilic acid is tocopherol succinate.

9. The metformin lipophilic acid salt of claim 1, wherein the lipophilic acid is tocopherol phosphate.

10. The metformin lipophilic acid salt of claim 1, wherein the lipophilic acid is a fatty acid.

11. The metformin lipophilic acid salt of claim 10, wherein the fatty acid has from about 8 to about 20 carbon atoms.

12. The metformin lipophilic acid salt of claim 1, wherein the lipophilic acid is oleic acid.

13. The metformin lipophilic acid salt of claim 1, wherein the lipophilic acid is stearic acid.

14. The metformin lipophilic acid salt of claim 1, wherein the lipophilic acid is lipoic acid.

15. The metformin lipophilic acid salt of claim 1, wherein the lipophilic acid is tocopherol nicotinic acid.

16. A pharmaceutical composition, comprising metformin, a lipophilic acid, and a pharmaceutically-acceptable carrier.

17. The composition of claim 16, wherein the composition is in the form of at least one of a solution, intravenous infusion, suspension, emulsion, gel, paste, powder, sustained release granules, sustained release coating, capsule, or tablet.

18. The composition of claim 16, wherein the pharmaceutically-acceptable carrier further comprises at least one of a salt, a buffering agent, a preservative, a solvent, or a therapeutic agent.

19. The composition of claim 16 further comprising at least one of sterile water, saline, D5W, or Ringer's solution.

20. The composition of claim 16, wherein the lipophilic acid is tocopherol succinate.

21. The composition of claim 16, wherein the lipophilic acid is tocopherol phosphate.

22. The composition of claim 16, wherein the lipophilic acid is tocopherol nicotinic acid.

23. The composition of claim 16, wherein the composition is in the form of a gel.

24. A method of making a metformin lipophilic acid salt, comprising combining metformin hydrochloride with a lipophilic acid in an aqueous base.

25. The method of claim 24, wherein the lipophilic acid is at least one of a tocopherol acid derivative or a fatty acid.

26. A method of producing a metformin gel, comprising:
(a) adding sodium chloride to a solution of tocopherol phosphate in water to provide a solution containing tocopherol phosphate and sodium chloride; and
(b) adding a metformin salt to the solution containing tocopherol phosphate and sodium chloride to provide a metformin salt solution.

27. The method of claim 26, wherein the metformin salt comprises metformin hydrochloride.

28. The method of claim 26, wherein the metformin salt comprises a metformin salt of a lipophilic acid.

29. The method of claim 28, wherein the lipophilic acid comprises a tocopherol acid derivative.

30. The method of claim 28, wherein the lipophilic acid comprises a tocopherol carboxylate.

31. The method of claim 28, wherein the lipophilic acid comprises a tocopherol phosphate.

32. The method of claim 28, wherein the lipophilic acid comprises a fatty acid.

33. A method for treating hyperglycemia in a warm-blooded animal, comprising administering to a warm-blooded animal a therapeutically effective amount of a pharmaceutical composition comprising a metformin salt of a lipophilic acid and a pharmaceutically-acceptable carrier.

34. The method of claim 33, wherein administering the composition provides anti-hyperglycemic activity.

35. The method of claim 33, wherein administering the composition comprises oral, rectal, parental, or intravenous administration.

36. The method of claim 33, wherein the composition further comprises a second therapeutic agent.

37. The method of claim 33, wherein the lipophilic acid comprises a tocopherol acid derivative.

38. The method of claim 33, wherein the lipophilic acid comprises a tocopherol carboxylate.

39. The method of claim 33, wherein the lipophilic acid comprises a tocopherol phosphate.

40. The method of claim 33, wherein the lipophilic acid comprises a fatty acid.

41. The method of claim 33, wherein the composition is in the form of a gel.

42. A method for decreasing the number of daily doses required to achieve a therapeutic effect in the treatment of hyperglycemia equivalent to that achieved when an eqimolar amount of metformin hydrochloride is administered, comprising administering an effective amount of a composition comprising a metformin salt of a lipophilic acid and a pharmaceutically-acceptable carrier.

43. The method of claim 42, wherein the lipophilic acid comprises a tocopherol acid derivative.

44. The method of claim 42, wherein the lipophilic acid comprises a tocopherol carboxylate.

45. The method of claim 42, wherein the lipophilic acid comprises a tocopherol phosphate.

46. The method of claim 42, wherein the lipophilic acid comprises a fatty acid.

47. The method of claim 42, wherein the composition is in the form of a gel.
